(19) United States
(12) Patent Application Publication
(10) Pub. No.: US 2023/0064711 A1
(43) Pub. Date: Mar. 2, 2023

(54) COMPOSITIONS, KITS AND METHODS FOR ENHANCING THERAPEUTIC COMPLIANCE

(71) Applicants: Michael BELKIN, Givat Shmuel (IL); RIVIA RESEARCH PTE. LTD., Singapore (SG)

(72) Inventor: Michael BELKIN, Givat Shmuel (IL)

(21) Appl. No.: 17/789,726

(22) PCT Filed: Dec. 30, 2020
(86) PCT No.: PCT/IL2020/051355

§ 371 Date: Jun. 28, 2022

(30) Foreign Application Priority Data
Dec. 30, 2019 (IL) 271763

(51) Int. Cl. A61K 31/5575; A61K 31/439; A61K 31/4178; A61K 31/27; A61K 31/4409; A61K 31/137; A61K 47/40; A61K 47/36; A61K 47/10; A61K 47/38; A61K 47/28; A61K 9/127; A61K 9/16; A61K 45/06

(52) U.S. Cl. CPC A61K 31/5575; A61K 31/439; A61K 31/4178; A61K 31/27; A61K 31/4409; A61K 31/137; A61K 47/40; A61K 47/36; A61K 47/10; A61K 47/38; A61K 47/28; A61K 9/127; A61K 9/1647; A61K 45/06

(57) ABSTRACT

The present disclosure encompasses pharmaceutical composition, kits and methods for enhancing therapeutic compliance.

### TECHNOLOGICAL FIELD

The present disclosure relates to compositions, kits and methods.

### BACKGROUND ART

References considered to be relevant as background to the presently disclosed subject matter are listed below:

[1] Schmidl D et al., J. Ocul. Pharmacol. Ther. 2015 March; 31(2):63-77

[2] Toris, C B et al., Am. J. Ophthalmol. 2001 June; 131:722-8.

[3] Diestelhorst, M., et al., Survey of ophthalmology. 2002; 47: S155-S161.

[4] Newman-Casey P N et al. Ophthalmology 2020; 127: 477-483.

Acknowledgement of the above references herein is not to be inferred as meaning that these are in any way relevant to the patentability of the presently disclosed subject matter.

### BACKGROUND

Glaucoma is an ocular disease associated with increased intra-ocular pressure. Anti glaucoma drugs that reduce the intra-ocular pressure act either by reducing aqueous humor production or by increasing the outflow of this fluid from the intra-ocular compartments [1]. Reducing the intraocular pressure reduces or arrests the progression of the disease and the irreversible progressive damage it inflicts on the optic nerve.

Previous reports show combinations of anti-glaucoma drugs with additional drugs directed to treatment of ocular disease [2, 3].

Reduced compliance with medication leads to acceleration of the glaucomatous degeneration processes and ensuing visual impairment [4].

### GENERAL DESCRIPTION

In accordance with some aspects, the preset disclosure relates to a pharmaceutical composition comprising at least one first compound associated with low compliance and at least one second compound associated with high compliance, the composition is for use in enhancing therapeutic compliance of the first compound.

In accordance with some other aspects, the preset disclosure relates to a pharmaceutical composition comprising at least one first compound capable of reducing intra-ocular pressure (IOP) and at least one other second compound capable of treating, preventing, inhibiting, reducing, eliminating, protecting or delaying the onset of an ocular condition having mid or severe symptoms.

In accordance with some other aspects, the preset disclosure relates to a modified release delivery system comprising a pharmaceutical composition comprising at least one first compound associated with low compliance and at least one second compound associated with high compliance, the composition is for use in enhancing therapeutic compliance of the first compound.

In accordance with another aspects, the preset disclosure relates to a delivery system allowing modified release (modified release delivery system) comprising a pharmaceutical composition comprising at least one first compound capable of reducing IOP and at least one other second compound capable of treating a different ocular condition having mid or severe symptoms.

In accordance with a further aspect, the preset disclosure relates to a kit comprising a first component and a second component, the first component comprises the at least one first compound capable of capable of reducing IOP and the second component comprises at least one second compound capable of treating, preventing, inhibiting, reducing, eliminating, protecting or delaying the onset of an ocular condition having mid or severe symptoms.

In accordance with some further aspects, the preset disclosure provides a method for enhancing therapeutic compliance of a first compound, the method comprising administering to an individual in need thereof, an effective amount of a pharmaceutical composition comprising at least one first compound associated with low compliance and at least one second compound associated with high compliance, to thereby enhance compliance of the first compound.

In accordance with some further aspects, the preset disclosure provides a method for enhancing therapeutic compliance of a treatment of an ophthalmic condition, the method comprising administering to an individual in need thereof, a pharmaceutical composition comprising an effective amount of at least one first compound capable of treating, preventing, inhibiting, reducing, eliminating, protecting or delaying the onset of an ophthalmic condition that requires reduction IOP, and an effective amount of at least one second compound capable of treating, preventing, inhibiting, reducing, eliminating, protecting or delaying the onset of an ocular condition having mild or severe symptoms.

In accordance with some further aspects, the preset disclosure provides a method of treating, preventing, inhibiting, reducing, eliminating, protecting or delaying the onset of a condition that requires reduction IOP, the method comprising administering to an individual in need thereof, a pharmaceutical composition comprising an effective amount of at least one first compound capable of treating, preventing, inhibiting, reducing, eliminating, protecting or delaying the onset of a condition that requires reduction IOP, and an effective amount of at least one second compound capable of treating, preventing, inhibiting, reducing, eliminating, protecting or delaying the onset of an ocular condition having mild or severe symptoms.

### DETAILED DESCRIPTION OF EMBODIMENTS

Therapeutic compliance has been a topic of clinical concern for more than 50 years due to the widespread nature of non-compliance with therapy requirements. There are several factors affecting therapeutic compliance including, inter alia, patient-related factors, therapy-related factors, healthcare system factors, social factors, economic factors, as well as disease-related factors.

Low compliance is observed in various diseases, including, inter alia lung disease, or ophthalmic disease, specifically chronic ophthalmic diseases associated with a need to reduce/lower intraocular pressure.

Accordingly, it was suggested to combine a treatment of a condition associated with low compliance with an additional treatment of a condition associated with mid to high compliance in order to increase the low compliance treatment and hence increase treatment efficacy of the condition to be treated by the low compliance treatment. As appreciated, enhancing therapy compliance may reduce health care costs and improve care quality as well as patient condition and health outcome.

While the condition to be treated with a first low compliance treatment has almost none or low symptoms and hence not fully treated, the condition to be treated with a second treatment has mid or severe symptoms which necessitate the treatment and hence the second treatment is considered as a high compliance treatment. Combining these two treatments would results in better treatment of the low compliance treatment.

For example and as described herein, it was suggested that the low compliance treatment of conditions associated, for example, with the need to reduce intra-ocular pressure (IOP), may be enhanced by combining these treatments with an additional high compliance treatment directed to a different condition, such as an ocular condition having mid to severe symptoms. The combination of a first treatment, including, inter alia, an anti-glaucoma treatment with an additional second high-compliance treatment, may assist in increasing compliance of the glaucoma treatment.

Hence, the present disclosure relates to a treatment combination for increasing/enhancing compliance of low compliance treatment by combining the low compliance treatment with a high compliance treatment.

Thus, in accordance with some aspects, the present disclosure provides, a treatment combination comprising a first treatment associated with low compliance and a second treatment associated with high compliance for use in increasing or enhancing therapeutic compliance of the first treatment.

The present disclosure also provides in accordance with some aspects, a method for enhancing compliance of treatment, the method comprising administering to an individual in need thereof, an effective amount of a first treatment and an effective amount second treatment having mild or severe symptoms to thereby enhance compliance of the first treatment.

Specifically, the present disclosure provides a method for enhancing compliance of a first compound, the method comprising administering to an individual in need thereof, an effective amount of a pharmaceutical composition comprising at least one first compound associated with low compliance and at least one second compound associated with high compliance, to thereby enhance compliance of the first compound.

In the following text, when referring to a composition it is to be understood as also referring to the kits, systems and methods disclosed herein. Thus, whenever providing a feature with reference to the composition, it is to be understood as defining the same feature with respect to the kits, systems and methods, mutatis mutandis.

The first treatment and the second treatment refer to any act or manner capable of treating, preventing, inhibiting, reducing, eliminating, protecting or delaying the onset of a condition as described herein and hence encompasses any treatment known in the art, including, inter alia, a drug, a medical device, an energy source or surgery.

The term “compliance” or “therapeutic/medication compliance” often used interchangeably with “adherence” or “concordance”, relates in the context of the present invention to a patient's behavior with respect with the recommendations of a healthcare provider and the extent by which the patient acts in accordance with the prescription. As used herein the term compliance encompasses also therapeutic/medication persistence and adequate usage.

Several parameters are considered when determining compliance to a prescribed treatment, for example at least one of the dose, dosing interval, duration of time from initiation to discontinuation of therapy or any combination thereof. Therapeutic non-compliance occurs when an individual's do not follow the recommendations as prescribed by a healthcare provider and detailed above.

The first compound is characterized by low compliance.

In the context of the present disclosure, low compliance is regarded as (i) treatment not taken by an individual in need thereof, (ii) treatment is taken by an individual in need thereof, but at a lower dose than prescribed, (iii) treatment is taken by an individual in need thereof at a prescribed dose but not at the prescribed frequency (i.e. longer dosing intervals), (iv) treatment is taken by an individual in need thereof but at a lower dose than prescribed and not at the prescribed frequency, (v) treatment is taken by an individual in need thereof at a prescribed dose and at the prescribed frequency but not for the entire prescribed duration (i.e. treatment is stopped prior to healthcare instructions), (vi) treatment is taken by an individual in need thereof but at a lower dose than prescribed and at the prescribed frequency and not for the entire prescribed duration, (vii) treatment is taken by an individual in need thereof but at a lower dose than prescribed and not at the prescribed frequency and not for the entire prescribed duration, (viii) treatment is taken by an individual in need thereof at a prescribed dose and not at the prescribed frequency and not for the entire prescribed duration, or (ix) any combinations thereof.

The term low compliance may be defined for example by a range of between 20% to about 95%, at times 40% to 80%, at times 50% to 80% of the treatment of the first not being followed as prescribed.

In other words, about 20% to 95%, at times 40% to 80%, at times 50% to 80% or any intermediate range, of the prescribed medications, such as anti-glaucoma medications, were not taken by the patient as prescribed in at least one of timing of treatment (i.e. duration of treatment), dosage and frequency (interval between sequential administrations). The outcome of the low compliance is as detailed herein, lack of adequate therapy.

In some embodiments, the low compliance is one or more of the following (i) not filling a received prescription, (ii) forgetfulness, (iii) multiple medications (such as eye drops), (iv) inability to instill the medication properly (e.g. the drops in the eye), (v) costs, availability, (vi) side effects or (vii) a combination thereof.

In contrast with the first treatment, the second treatment is associated with high compliance. A patient suffering from the condition treatable by the second treatment (i.e. compound) being characterized by high compliance, is expected to follow the recommendations as prescribed by a healthcare provider. In other words, the condition to be treated by the second drug is characterized by having disease symptoms that necessitate treatment (either mild symptoms or sever symptoms).

The term high compliance indicates that between 55% to about 100%, at times 60% to 90%, at times 70% to 85% of the prescribed drugs were taken as prescribed.

It was suggested by the inventor that by combining both the first compound and the second compound, the patient in need of the second compound, will use the first compound as well.

Enhancing (increasing) the compliance by the invention encompasses increasing medication persistence, include at least one of the factors detailed herein above in connection with the definition of low compliance, for example maintaining the dosing regimen with respect to the dose, maintaining the dosing interval (frequency) as well as increasing the duration of time from initiation to discontinuation of therapy.

The enhanced compliance of the invention may be determined by various measures.

For example, compliance may be measured using either process-oriented or outcome-oriented.

Process-oriented indicators make use of intermediate variables such as appointment-keeping or pill counts while outcome-oriented definitions use the end-result of treatment, e.g. cure rate, as an indicator of success.

In some embodiments, the enhanced therapeutic compliance may be determined by the ability of a patient's to follow the prescription of a healthcare provider.

In some embodiments, the enhanced therapeutic compliance may be determined by a patient following at least one of dosing regimen with respect to the dose, maintaining the dosing interval as well as increasing the duration of time from initiation to discontinuation of therapy.

In some embodiments, the enhanced therapeutic compliance may be determined by electronic means that are included in the composition container and send information when the applicator is in use.

In some embodiments, the enhanced therapeutic compliance may be determined by the therapeutic effect (outcome) of the first compound and therapy success. The enhanced therapeutic effect may be reflected by at least one of achieving a desired therapeutic effect of at least one of the drugs, achieving a desired therapeutic effect of at least one anti-glaucoma drug.

The enhanced compliance may be determined on a population-based study or on an individual-based study.

In some embodiments, the first treatment and the second treatment is each a drug, specifically a different drug.

A drug encompasses any compound such as a chemical compound, a biological compound or a combination thereof. Hence, the first treatment refers to a first compound and the second treatment refers to a second compound.

For the purpose of increasing compliance of the first compound, the first compound and second compound may be administered together, as disclosed herein.

In some embodiments, the first compound and the second compound are administered concomitantly.

In some embodiments, the two compounds are administered (at times prior to administration) in a single unit dose; namely in a unit which is suitable for administration to the subject (human or non-human) as detailed herein below. The unit dose can contain an effective amount−a prescribed quantity of the first compound and the second compound sufficient to produce a therapeutic effect.

Hence, the present invention also provides a medicament or a pharmaceutical composition, comprising a combination of the first compound and the second compound and further optionally at least one carrier, excipient or an additive. Where the pharmaceutical composition is used, one can utilize the effect, e.g. high compliance of the second compound and use it to increase the low compliance of the first compound.

In some embodiments, the pharmaceutical composition comprising the first compound and the second compound together for simultaneous administration.

A “pharmaceutical composition” of the invention comprises a therapeutically effective amount of the first compound and the second compound together, optionally with suitable additives such as diluents, preservatives, solubilizers, emulsifiers, adjuvant and/or carriers.

The pharmaceutical composition comprising the first compound and the second compounds are as described herein for use in increasing treatment compliance of a low compliance first compound.

Hence, in accordance with some aspects, the present disclosure provides a pharmaceutical composition comprising at least one first compound associated with low compliance and a second compound associated with high compliance for use in enhancing therapeutic compliance of the first compound.

As described herein and in accordance with some embodiments, the first treatment and the second treatment are related to ocular disease, specifically the first treatment is for use in treating a condition that requires reduction in intra-ocular pressure (IOP).

Hence, the pharmaceutical composition comprises a combination of at least one first compound capable of treating, preventing, inhibiting, reducing, eliminating, protecting or delaying the onset of a condition that requires reduction IOP, and at least one second compound capable of treating, preventing, inhibiting, reducing, eliminating, protecting or delaying the onset of an ocular condition having mid or severe symptoms.

In accordance with some aspects, the present disclosure provides, a pharmaceutical composition for use in enhancing therapeutic compliance of a treatment capable of treating, preventing, inhibiting, reducing, eliminating, protecting or delaying the onset of a chronic ophthalmic diseases, especially these that requires reduction IOP. The composition comprises at least one first compound capable of treating, preventing, inhibiting, reducing, eliminating, protecting or delaying the onset of a condition that requires reduction IOP, and at least one second compound capable of treating, preventing, inhibiting, reducing, eliminating, protecting or delaying the onset of an ocular condition having mid or severe symptoms.

In some embodiments, by increasing compliance of the first treatment, specifically the first compound, the treatment efficacy of the first treatment is enhanced and hence increases the treating, preventing, inhibiting, reducing, eliminating, protecting or delaying the onset the condition to be treated by the first treatment, specifically condition that requires reduction IOP, such as glaucoma.

IOP refers to the fluid pressure inside the eye. Increased (elevated) IOP is associated with anatomical and histological damage to the eye, specifically irreversible and untreatable damage to the optic nerve which, is unchecked, leads to progressive visual field loss, visual impairment and blindness. Ocular hypertension (OHT) is defined by intraocular pressure being higher than normal in the absence of optic nerve damage or visual field loss, where the normal IOP is generally defined to be between 10 mmHg and 20 mmHg As appreciated, IOP may be measured with a tonometer, typically during an eye examination or after surgery.

In some embodiments, the pharmaceutical composition comprises a first treatment, specifically, the first compound is capable of reducing/lowering intra-ocular pressure (IOP).

While, the condition to be treated by the first compound has hardly no symptoms (e.g. glaucoma), the second compound is selected to treat a different ocular condition (disease, disorder, i.e. not associated with increased IOP) characterized by having mid or severe symptoms. Mid or severe symptom is to be understood as any change from normal function or feeling that is apparent in an individual (patient), the symptom(s) typically reflect the presence of an unusual state, or of a condition, disease or disorder.

The second compound is typically not used as first-line treatment to treat chronic ophthalmic diseases associated with increased intraocular pressure.

As detailed herein, the pharmaceutical composition comprises a first compound capable of treating, preventing, inhibiting, reducing, eliminating, protecting or delaying the onset of a condition associated with a requirement to reduce IOP (i.e. in need to reduce IOP or that require reduction of IOP) and a second compound characterized by high compliance.

In some further embodiments, the first treatment is for use in treating, preventing, inhibiting, reducing, eliminating, protecting or delaying the onset of a chronic ophthalmic disease, specifically a disease associated with reduction of IOP.

In some embodiments, the first compound is for use in treating, preventing, inhibiting, reducing, eliminating, protecting or delaying the onset of condition associated with increased IOP.

Increased IOP is an important risk factor for glaucoma.

In some other embodiments, the first compound is for treating, preventing, inhibiting, reducing, eliminating, protecting or delaying the onset of glaucoma.

Glaucoma, typically leads to a loss of vision and is considered as one of the leading cause of blindness after cataracts and the most common cause of irreversible blindness as vision loss from glaucoma, once it has occurred, is untreatable and permanent. It has no significant early symptoms or pain and is typically diagnosed during a comprehensive eye examination. Patients suffering from early or mild glaucoma often do not experience disease symptoms (i.e. they experience almost normal feeling or function) and hence, despite the availability of multiple glaucoma therapies and the required long-term treatment, such therapies are not used as prescribed, resulting in low compliance and progression of the irreversible optic nerve damage caused by the disease. It causes damage to the optic nerve due to a number of factors, including high IOP, excessive fluid pressure within the eye, which can be due to various reasons including blockage of drainage ducts, and narrowing or closure of the angle between the iris and cornea.

The term glaucoma refers to a group of eye conditions (disease) such as open-angle glaucoma, closed-angle glaucoma and normal-tension glaucoma.

Open-angle glaucoma is the most common type, in which the drainage angle for fluid within the eye remains open. This type of glaucoma develops slowly over time and is painless and does not have acute attacks. Thus, it can be diagnosed only by screening via regular eye check-ups. The only signs are gradually progressive visual field loss, peripheral vision may begin to decrease, followed by central vision, resulting in blindness if its progression is not stopped by reducing the IOP.

Closed-angle glaucoma and normal-tension glaucoma are less common types of glaucoma. Closed-angle glaucoma can present either gradually as in open angle glaucoma or suddenly. The sudden presentation may involve severe eye pain, blurred vision, mid-dilated pupil, redness of the eye, and nausea.

The first compound can be an existing glaucoma drug as well as such drug that are in development to be future drugs, all with the aim of enhancing treatment compliance.

In some embodiments, the pharmaceutical composition comprising an anti-glaucoma treatment with an additional second high-compliance treatment. In some other embodiments, the pharmaceutical composition may assist in increasing compliance of the anti-glaucoma treatment.

In some embodiments, the composition is for use in enhancing compliance of a treatment for a condition treatable by the first compound.

In some embodiments, the composition is for use in enhancing compliance of treatment in an individual suffering from a condition in which reduction of IOP is required.

In some embodiments, the composition is for use in enhancing compliance of treatment in an individual suffering from a condition associated with an increased IOP.

In some embodiments, the composition is for use in enhancing compliance of a treatment in an individual suffering from glaucoma.

In some embodiments, the composition is for use in enhancing compliance of a treatment in an individual diagnosed with glaucoma.

In some embodiments, the composition is for use in enhancing compliance of a treatment capable of treating glaucoma.

In some embodiments, the glaucoma is an open-angle glaucoma, closed-angle glaucoma, normal-tension glaucoma or any other of the less common types of glaucoma such as traumatic glaucoma.

In some embodiments, the glaucoma is an open-angle glaucoma, closed-angle glaucoma or normal-tension glaucoma.

In some embodiments, the composition is for use in enhancing compliance of a treatment in an individual suffering or diagnosed from open-angle glaucoma

In some embodiments, the first compound characterized by having low compliance is for use in treating, preventing, inhibiting, reducing, eliminating, protecting or delaying the onset of glaucoma, specifically open-angle glaucoma.

As described herein, the combination of the low compliance treatment, such as anti-glaucoma treatment, can be enhanced by combing it with an additional treatment directed to a different condition, such as an ocular condition. The different condition, unlike for example glaucoma, has mid or severe symptoms that necessitate the treatment and hence the treatment is considered as a high compliance.

In some embodiments, the second treatment, specifically the second compound is capable of treating, preventing, inhibiting, reducing, eliminating, protecting or delaying the onset of an ocular condition having mid or severe symptoms.

In some embodiments, the second compound is typically not used to treat a chronic ophthalmic disease associated with the need to reduce IOP.

In the context of the present disclosure reference to a compound it is to be understood as referring to a pharmaceutically acceptable salt thereof, solvate thereof, hydrate thereof, any stereoisomer therefore, physiologically functional derivative thereof or any combination thereof.

In some embodiments, the first compound is at least one of a prostaglandin analogue (PGA), a beta-adrenergic receptor antagonist, an alpha2-adrenergic agonist, an alpha agonist, a miotic agent, carbonic anhydrase inhibitor, nitric oxide, a rho kinase inhibitor or any combination thereof.

A prostaglandin analogue as used herein refers to a class of drugs (compounds) that bind to a prostaglandin receptor. In the field of ophthalmology, a prostaglandin analogue is typically used to lower IOP in individuals diagnosed with a condition in need to reduce IOP, specifically, glaucoma. A prostaglandin analogue typically increases the outflow of aqueous humor, mainly by relaxing the ciliary muscle, and possibly also due to changes in extracellular matrix and to widening of spaces within the trabecular meshwork.

In some embodiments, the first compound is a PGA.

In some embodiments, the PGA is one or more of latanoprost (isopropyl analogue of PGF2α), bimatoprost, travoprost, tafluprost, latanoprostene bunod, any pharmaceutically acceptable salt thereof, solvate thereof, hydrate thereof, any stereoisomer therefore, physiologically functional derivative thereof or any combinations thereof.

In some other embodiments, the PGA is bimatoprost or any pharmaceutically acceptable salt thereof, solvate thereof, hydrate thereof, any stereoisomer therefore, physiologically functional derivative thereof.

Bimatoprost as used herein refers to a compound having the following structure:

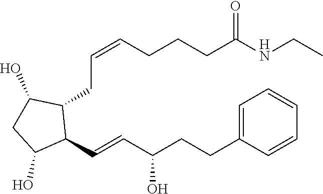 [SMILES: CCNC(=O)CCC/C=C\C[C@@H]1[C@@H](/C=C/[C@@H](O)CCc2ccccc2)[C@H](O)C[C@@H]1O]

In some other embodiments, the PGA is at least one of latanoprost, travoprost, tafluprost or any pharmaceutically acceptable salt thereof, solvate thereof, hydrate thereof, any stereoisomer therefore, physiologically functional derivative thereof or any combinations thereof.

In some other embodiments, the PGA is travoprost or any pharmaceutically acceptable salt thereof, solvate thereof, hydrate thereof, any stereoisomer therefore, physiologically functional derivative thereof. Travoprost as used herein refers to a compound having the following structure:

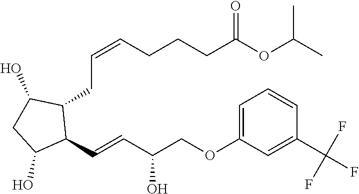 [SMILES: CC(C)OC(=O)CCC/C=C\C[C@@H]1[C@@H](/C=C/[C@@H](O)COc2cccc(C(F)(F)F)c2)[C@H](O)C[C@@H]1O]

In some other embodiments, the PGA is tafluprost or any pharmaceutically acceptable salt thereof, solvate thereof, hydrate thereof, any stereoisomer therefore, physiologically functional derivative thereof. Tafluprost as used herein refers to a compound having the following structure:

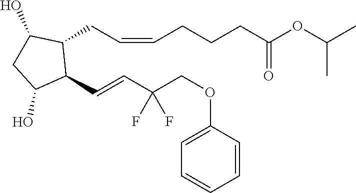 [SMILES: CC(C)OC(=O)CCC/C=C\C[C@@H]1[C@@H](/C=C/C(F)(F)COc2ccccc2)[C@H](O)C[C@@H]1O]

In some other embodiments, the PGA is latanoprost or any pharmaceutically acceptable salt thereof, solvate thereof, hydrate thereof, any stereoisomer therefore, physiologically functional derivative thereof.

Latanoprost as used herein refers to a compound having the following structure:

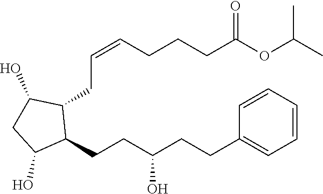 [SMILES: CC(C)OC(=O)CCC/C=C\C[C@@H]1[C@@H](CC[C@@H](O)CCc2ccccc2)[C@H](O)C[C@@H]1O]

In some embodiments, PGA is latanoprostene bunod (Vyzulta) or any pharmaceutically acceptable salt thereof, solvate thereof, hydrate thereof, any stereoisomer therefore, physiologically functional derivative thereof.

Latanoprostene bunod (Vyzulta) refers to a compound that is metabolized into latanoprost and nitric oxide.

Latanoprostene bunod s used herein refers to a compound having the following structure:

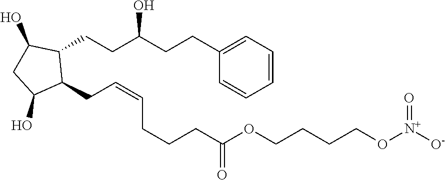 [SMILES: O=C(CCC/C=C\C[C@@H]1[C@@H](CC[C@@H](O)CCc2ccccc2)[C@H](O)C[C@@H]1O)OCCCCO[N+](=O)[O-]]

A beta-adrenergic receptor antagonist as used herein refers to a medication that decrease aqueous humor production by the epithelium of the ciliary body.

In some embodiments, a beta-adrenergic receptor antagonist is timolol, levobunolol, betaxolol or any combination thereof.

An alpha2-adrenergic agonist refers to a medication that decreases aqueous humor production and increasing uveoscleral outflow.

In some embodiments, an alpha2-adrenergic agonist is brimonidine, apraclonidine or any combination thereof.

An alpha agonist refers to typically a less selective medication, that decrease aqueous humor production through vasoconstriction of ciliary body blood vessels.

In some embodiments an alpha agonist is epinephrine.

A miotic agent refer to a medication that is effective by contraction of the ciliary muscle, opening the trabecular meshwork and allowing increased outflow of the aqueous humor.

In some embodiments a miotic agent is pilocarpine, echothiophate or any combinations thereof.

A carbonic anhydrase inhibitor refers to a medication which lower secretion of aqueous humor by inhibiting carbonic anhydrase in the ciliary body.

In some embodiments a carbonic anhydrase inhibitor is dorzolamide, brinzolamide, acetazolamide or any combinations thereof.

In some embodiments, the first compound is Vyzultatm® or Rhopressa®.

As appreciated, Vyzultatm® is a PGA that is metabolized into 2 moieties and regulates IOP through both the trabecular outflow and uveoscleral outflow pathways.

In some embodiments, the first compound is nitric oxide.

The composition also comprises an additional compound, denoted herein as a second compound. As detailed herein, the second compound is capable of treating, preventing, inhibiting, reducing, eliminating, protecting or delaying the onset of an ocular condition characterized by mild or severe symptoms that results in high compliance of the drug.

In some embodiments, the mild or severe symptom may be at least one of inability to see near object clearly such as the need for reading glasses (presbyopia), blurred vision, eye pain stinging or itching, eye redness, eye discharge, light sensitivity, pupil construction, itching, irritation, dry eye syndrome, fatigued eyes, eye lids and lashes discoloration, mydriasis, inflammation, pain or any combination thereof.

In some embodiments, the condition to be treated by the second compound is at least one of blurred vision, presbyopia, itching (irradiated) eyes, eye redness, mydriasis, inflammation, dry eye syndrome or combination thereof.

In some embodiments, the condition to be treated by the second compound is presbyopia, itchy (irradiated) eyes, dry eye syndrome or combination thereof.

In some embodiments, the condition to be treated by the second drug is mydriasis.

Mydriasis refers to dilation of the pupil, either by a non-physiological cause (such as disease or trauma) or due to a physiological pupillary response.

In some embodiments, the condition to be treated by the second drug is presbyopia.

Presbyopia refers to a condition associated with the aging of the eye that results in progressively worsening ability to focus clearly on close objects. Symptoms include difficulty reading small print, having to hold reading material farther away, headaches, and eyestrain.

Hence, an individual suffering from presbyopia, would typically have to use appropriate eyeglasses or take appropriate medication in order to be able to read, hence resulting in high compliance to treatment.

In some embodiments, the second compound is one or more of pilocarpine, carbachol, aceclidine, tropicamide, phenylephrine, any pharmaceutically acceptable salt thereof, solvate thereof, hydrate thereof, any stereoisomer therefore, physiologically functional derivative thereof or combination thereof.

In some embodiments, the second compound is one or more of pilocarpine, aceclidine, tropicamide, phenylephrine, any pharmaceutically acceptable salt thereof, solvate thereof, hydrate thereof, any stereoisomer therefore, physiologically functional derivative thereof or combination thereof.

In some embodiments, the second compound is of pilocarpine or any pharmaceutically acceptable salt thereof, solvate thereof, hydrate thereof, any stereoisomer therefore, physiologically functional derivative thereof or combination thereof.

Pilocarpine as used herein refers to a compound having the following structure:

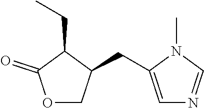

In some embodiments, the condition to be treated by the second compound is itchy eyes. Itchy eyes refer to a condition often is accompanied by itchy eyelids, especially at the base of the eyelashes and red eyes or swollen eyelids. Itchy eyes are typically caused by some type of allergy or as a side effect of preservatives in chronic eye drops.

In some embodiments, the second compound is naphazoline or any pharmaceutically acceptable salt thereof, solvate thereof, hydrate thereof, any stereoisomer therefore, physiologically functional derivative thereof.

In some embodiments, the second compound is a cannabinoid, preferably a cannabinoid having anti-nociceptive or anti-inflammatory properties.

In some embodiments, the second compound is nepafenac or any pharmaceutically acceptable salt thereof, solvate thereof, hydrate thereof, any stereoisomer therefore, physiologically functional derivative thereof.

For purposes of the present disclosure, the term “cannabinoid” includes any member of naturally occurring and synthetic cannabinoids and related compounds, and extracts from any Cannabis species and varieties. The cannabinoids may be natural, semisynthetic, or synthetic. They may be included in its free form, or in the form of a salt; an acid addition salt of an ester; an amide; an enantiomer; an isomer; a tautomer; a prodrug; different isomeric forms (for example, enantiomers and diastereoisomers), both in pure form and in admixture, including racemic mixtures.

In some embodiments, the second compound is cannabidiol (CBD) or any pharmaceutically acceptable salt thereof, solvate thereof, hydrate thereof, any stereoisomer therefore, physiologically functional derivative thereof or combination thereof.

In some embodiments, the condition to be treated by the second drug is dry eye syndrome (DES) known as keratoconjunctivitis sicca (KCS). DES is a condition of having dry eyes due to malfunctioning of the tear secretion apparatus.

In some embodiments, the second compound is at least one of a supplemental lubrication, autologous serum, a steroid or combination thereof.

In some embodiments, the composition of the invention comprises at least one PGA or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition or kits of the invention comprises one or more of latanoprost, bimatoprost, travoprost, tafluprost, latanoprostene bunod, any pharmaceutically acceptable salt thereof, solvate thereof, hydrate thereof, any stereoisomer therefore, physiologically functional derivative thereof or combinations thereof and pilocarpine or any pharmaceutically acceptable salt thereof, solvate thereof, hydrate thereof, any stereoisomer therefore, physiologically functional derivative thereof.

In some embodiments, the composition or kits of the invention comprises latanoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition or kits of the invention comprises tafluprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition and kits of the invention comprises travoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition of the invention comprises bimatoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition of the invention comprises latanoprostene bunod or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the composition of the invention comprises at least nitric oxide or any pharmaceutically acceptable salt thereof, solvate thereof, hydrate thereof, any stereoisomer therefore, physiologically functional derivative thereof.

The term “pharmaceutically acceptable salt” refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of a compound described herein.

The term “solvate” refers to an aggregate of a molecule with one or more solvent molecules, such as hydrate, alcoholate (aggregate or adduct with alcohol), and the like.

The compounds of the present invention, as defined above, may have the ability to crystallize in more than one form, a characteristic, which is known as polymorphism, and it is understood that such polymorphic forms (“polymorphs”) are within the scope of formulae described herein above. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

The term “hydrate” refers to a compound formed by the addition of water. The hydrates may be obtained by any known method in the art by dissolving the compounds in water and recrystallizing them to incorporate water into the crystalline structure.

A “pharmaceutically acceptable prodrug” is a compound that may be converted under physiological conditions to the specified compound or to a pharmaceutically acceptable salt of such compound.

The term “physiologically functional derivative” used herein relates to any physiologically acceptable derivative of a compound as described herein. The physiologically functional derivatives also include prodrugs of the compounds of the invention. Such prodrugs may be metabolized in vivo to a compound of the invention. These pro-drugs may or may not be active themselves and are also an object of the present invention.

The amount of each compound in the composition and kits may vary depending on the specific nature of the at least two compounds (i.e. the first compound and the second compound) and the patient's condition and may be adjusted accordingly. One of parameters that may vary is the ratio of the two compounds in the composition.

In some embodiments the composition comprise the first compound and the second compound at a ratio selected from 50:1, 20:1, 10:1, 8:1, 7:1, 6:1, 5:1, 4:1, 3:1, 2:1, 1:1, 1:1.5, 1:2, 1:2.5, 1:3, 1:4, 1:5. 1:6, 1:7, 1:8, 1:10, 1:20 or 1:50.

In some embodiments, the pharmaceutical composition comprising at least 0.001 w/w % of the first compound and at least 0.05 w/w % of the second compound. In some other embodiments, the pharmaceutical composition comprising between about 0.001% and about 95% of the first compound and a between about 0.05% and about 95% of the second compound.

In some embodiments, the pharmaceutical composition comprising the first compound between about 0.001% w/w and about 95% w/w, between about 0.001% and about 90%, between about 0.001% and about 85%, between about 0.001% and about 80%, between about 0.001% and about 75%, between about 0.001% and about 70%, between about 0.001% and about 65%, between about 0.001% and about 60%, between about 0.001% and about 55%, between about 0.001% and about 50%, between about 0.001% and about 45%, between about 0.001% and about 40%, between about 0.001% and about 35%, between about 0.001% and about 30%, between about 0.001% and about 25%, between about 0.001% and about 20%, between about 0.001% and about 15%, between about 0.001% and about 10%, at times between about 0.005% to about 10% in the composition, at times between about 0.01% to about 6% in the composition, at times between about 0.05% to about 6% in the composition, at times between about 0.1% to about 6% in the composition, at times between about 0.5% to about 6% in the composition, at times between about 0.001% to about 5% in the composition, at times between about 0.005% to about 2% in the composition, at times between about 0.005% to about 0.5% in the composition, at times between about 0.005% w/w to about 0.1 w/w % in the composition (all % are provided in w/w) and the second compound between about 0.05% w/w and about 95% w/w, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10% in the composition, at times between about 0.2% to about 5% in the composition, at times between about 0.5% to about 5% in the composition, at times between about 1% to about 5% in the composition, at times between about 2% to about 5% in the composition, at times between about 0.1% to about 3% in the composition, at times between about 0.1% w/w to about 1% w/w in the composition (all % are provided in w/w) in any combination thereof.

In some embodiments, the pharmaceutical composition comprising the first compound between about 0.001% w/w and about 95% w/w, between about 0.001% and about 90%, between about 0.001% and about 85%, between about 0.001% and about 80%, between about 0.001% and about 75%, between about 0.001% and about 70%, between about 0.001% and about 65%, between about 0.001% and about 60%, between about 0.001% and about 55%, between about 0.001% and about 50%, between about 0.001% and about 45%, between about 0.001% and about 40%, between about 0.001% and about 35%, between about 0.001% and about 30%, between about 0.001% and about 25%, between about 0.001% and about 20%, between about 0.001% and about 15%, between about 0.001% and about 10%, at times between about 0.005% to about 10% in the composition, at times between about 0.01% to about 6% in the composition, at times between about 0.05% to about 6% in the composition, at times between about 0.1% to about 6% in the composition, at times between about 0.5% to about 6% in the composition, at times between about 0.001% to about 5% in the composition, at times between about 0.005% to about 2% in the composition, at times between about 0.005% to about 0.5% in the composition, at times between about 0.005% w/w to about 0.1 w/w % in the composition (all % are provided in w/w).

In some embodiments, the pharmaceutical composition comprising the second compound between about 0.05% w/w and about 95% w/w, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10% in the composition, at times between about 0.2% to about 5% in the composition, at times between about 0.5% to about 5% in the composition, at times between about 1% to about 5% in the composition, at times between about 2% to about 5% in the composition, at times between about 0.1% to about 3% in the composition, at times between about 0.1% w/w to about 1% w/w in the composition (all % are provided in w/w).

In some other embodiments, the pharmaceutical composition comprising between about 0.001% and about 95% of a PGA and a between about 0.05% and about 95% of pilocarpine, any pharmaceutically acceptable salt thereof, solvate thereof, hydrate thereof, any stereoisomer therefore or physiologically functional derivative thereof.

In some other embodiments, the pharmaceutical composition comprising between about 0.001% and about 95% of one or more of latanoprost, bimatoprost, travoprost, tafluprost, latanoprostene bound, any pharmaceutically acceptable salt thereof, solvate thereof, hydrate thereof, any stereoisomer therefore, physiologically functional derivative thereof or any combinations thereof and a between about 0.05% and about 95% of pilocarpine, any pharmaceutically acceptable salt thereof, solvate thereof, hydrate thereof, any stereoisomer therefore or physiologically functional derivative thereof.

In some other embodiments, the pharmaceutical composition comprising between about 0.001% and about 95% of one or more of tafluprost, latanoprost, travoprost or combination thereof and a between about 0.05% and about 95% of pilocarpine.

In some other embodiments, the pharmaceutical composition comprising between about 0.001% and about 10% of a PGA and a between about 0.05% and about 10% of pilocarpine, any pharmaceutically acceptable salt thereof, solvate thereof, hydrate thereof, any stereoisomer therefore or physiologically functional derivative thereof.

In some other embodiments, the pharmaceutical composition comprising between about 0.001% and about 10% of one or more of latanoprost, bimatoprost, travoprost, tafluprost, latanoprostene bound, any pharmaceutically acceptable salt thereof, solvate thereof, hydrate thereof, any stereoisomer therefore, physiologically functional derivative thereof or any combinations thereof and a between about 0.05% and about 10% of pilocarpine, any pharmaceutically acceptable salt thereof, solvate thereof, hydrate thereof, any stereoisomer therefore or physiologically functional derivative thereof.

In some embodiments, the pharmaceutical composition comprising between about at least 0.01% (w/w) bimatoprost. In some embodiments, the composition comprising between about 0.01% w/w and 10% w/w bimatoprost.

In some embodiments, the pharmaceutical composition comprising between about at least 0.0015% (w/w) tafluprost. In some embodiments, the composition comprising between about 0.015% w/w and 10% w/w tafluprost.

In some embodiments, the pharmaceutical composition comprising between about at least 0.005% (w/w) latanoprost. In some embodiments, the composition comprising between about 0.005% w/w and 10% w/w latanoprost.

In some embodiments, the pharmaceutical composition comprising between about at least 0.004% (w/w) travoprost. In some embodiments, the composition comprising between about 0.004% w/w and 10% w/w travoprost.

In some embodiments, the pharmaceutical composition comprising between about at least 0.5% (w/w) pilocarpine. In some embodiments, the composition comprising between about 0.5% w/w and 10% w/w pilocarpine.

The amounts of the first compound and of the second compound, can according with some embodiments, have a synergistic effect. Hence, according with some embodiments, the composition is considered as a synergistic composition or a. The amount of the first compound and of the second compound, in accordance with some embodiments are synergistic amounts. Synergy can be determined by known methods in the art.

The pharmaceutical compositions may be prepared in a dosage form that is suitable to be administrated topically to the conjunctiva, the cornea or the eyelid or administrated parenterally, e.g. intraocular injection to the anterior, posterior and vitreous chambers.

In some embodiments, the composition is designed to be administered topically to the surface of the eye. In some embodiments, the composition is designed to be administered to the posterior segment of the eye. The main features which are of interest for topical delivery are conjunctiva, cornea or lacrimal fluid.

The conjunctiva is located at the outside of the eye and joins on to the cornea and eyelids. The cornea is non-vascular and negatively charged and is composed of three layers: epithelium; stroma and endothelium. The lacrimal fluid is secreted from glands and is located on the surface of the eye and has a pH of 7.4.

The composition may be applied to the eye by any known application method in the art, for local administration. In some embodiments, administration of the composition is by local administration.

In some embodiments, the composition is administered topically. Hence the composition is adjusted to be suitable for topical administration. “Topical administration” and “administered topically” as used herein mean that the administration of the composition comprising the first compound and the second compound or a kit comprising the first compound and the second compound is applied to a particular place on or in the body. Specifically, the composition or kit of the invention is each considered to be administered onto the eye, more specifically to the conjunctiva.

The topically administrable compositions may be formulated into a suitable formulation or composition with at least one carrier.

As described herein below, the carriers may be selected from powders, oils, creams, foams, ointments, lotions, gels, pastes, mousiness, hydrogels or combination thereof. In some other embodiment, the composition is in the form of a solution, a suspension, a paste, a cream, a foam, gel or an ointment.

In some embodiment, the composition is an ocular solution or an ocular suspension. In some embodiment, the composition is an aqueous solution or an aqueous suspension. In some embodiment, the composition is in the form of eye drops, eye spray or eye cream.

In some embodiments, the composition is in the form of eye drops of a suspension or solution. In some embodiments, the composition is applied to the eye in a form of topical drop. The eye drops may be in isotonic, pH-adjusted, sterile saline. Administration of the eye drops into the eye may be using a dropper, or a container with a dropper nozzle or a tube with a nozzle.

In some embodiments, the composition is an ocular solution. The term solution as used herein encompasses a range of viscosities, ranging from low viscosity solution to high viscosity solutions (forming a gel-like solution).

The pH of ocular composition is an important feature for controlling for example the ocular acceptability of the composition and the absorption of the compound across the cornea. Ideally the pH of the composition should be adjusted to maximize the chemical stability and/or absorption of the compounds (the first compound and the second compound). In some embodiments, the pH of the ocular composition is about 7.4 as this is the pH of tear fluid.

The pharmaceutical composition may comprise a buffering agent, an agent which adjusts the osmolarity thereof, and optionally, one or more pharmaceutically acceptable carriers, excipients and/or additives as known in the art.

In some embodiments, the additive is at least one of a viscosity enhancer, permeation enhancer, cyclodextrins or a combination thereof. In some embodiments, the composition comprises at least one viscosity enhancer.

A viscosity enhancer typically improves precorneal residence time and bioavailability upon topical drop administration by enhancing formulation viscosity. The viscosity enhancer (modifier) may be added to control the rate at which the drop flows out of the container (and thus enhance ease of application); and, to control the residence time of the solution within the precorneal environment.

In some embodiments, the viscosity enhancer is a hydrophilic polymer.

In some embodiments, the viscosity enhancer is hydroxy methyl cellulose, hydroxy ethyl cellulose, sodium carboxy methyl cellulose, hydroxypropyl methyl cellulose (HMPC), poly(vinyl alcohol), poly(acrylic acid) or any combination thereof.

The amount of the viscosity enhancer is such that it is added to enhance the viscosity of ocular composition.

In some embodiments, the composition comprising at least one of polyethylene glycol, polyvinyl alcohol, propylene glycol, carboxymethylcellulose, povidone, glycerin, mineral oil or combination thereof.

In some embodiments, the composition comprises HPMC. In some embodiments, the composition comprises HPMC in a concentration of between about 0.45 and about 1.0% w/w.

In some embodiments, the composition comprises polyvinyl alcohol. Polyvinyl alcohol is a water-soluble vinyl polymer and may be used in the composition as either one of high-viscosity (average molecular weight 200 000 g/mol); medium-viscosity (average molecular weight 130 000 g/mol) or low-viscosity (average molecular weight 20 000 g/mol). In some embodiments, the composition comprises poly(vinyl alcohol) in a concentration of between about 0.25% and about 3.00% w/w.

In some embodiments, the composition comprises polyacrylic acid.

In some embodiments, the composition comprises at least one permeation enhancer. A permeation enhancer typically improves corneal uptake by modifying the corneal integrity.

In some embodiments, the permeation enhancer is a chelating agent, a preservative, a surface-active agent, bile salt or any combination thereof.

In some embodiments, the permeation enhancer is benzalkonium chloride, polyoxyethylene glycol ethers (lauryl, stearyl and oleyl), ethylenediaminetetra acetic acid sodium salt, sodium taurocholate, saponins, cremophor EL or any combination thereof.

In some embodiments, the composition is an ocular suspension. The use of suspensions may be required for compound that have problems with chemical stability or solubility.

In some embodiments the composition comprises surfactant (e.g. poloxamer), water, buffer, thickener, preservative, and chitosan.

Depending on the specific compounds to be used in the composition and the intended use of the compounds, there may be a need to control the release of the first compound and/or the second compound from the dosage from.

In some embodiments, the composition comprises a delivery system.

As used herein a delivery system is a formulation or a device that enables the introduction of a therapeutic substance in the body and improves its efficacy and safety by controlling, for example, the rate, time and place of release of drugs in the body. Such delivery systems typically control the rate at which a drug is released and the location in the body where it is released. Some systems can control both.

In some embodiments, the delivery system is one or more of liposome, niosome, microsponge, microemulsion, microsphere, solid lipid nanoparticles (SLN), aerosol or combination thereof.

For some applications as further described herein, modified release of the compounds of such delivery systems may be essential.

In some embodiments, the composition may be in a form of a dosage form designed to release at least one of the two compounds, the first compound and/or the second compound at a controlled release mode, which may be a slow release mode or a rapid release mode.

In some embodiments, the composition is a solution providing a pulse permeation of the first compound and/or of the second compound post topical administration, after which the concentration of the first compound and/or of the second compound declines.

In accordance with some aspects, the present disclosure provides a modified release delivery system comprising the composition of the invention.

Specifically, it is provided a modified release delivery system for enhancing therapeutic compliance of a treatment capable of treating, preventing, inhibiting, reducing, eliminating, protecting or delaying the onset of a chronic ophthalmic diseases associated with low compliance.

Specifically, the modified release delivery system comprises the composition comprising a first compound characterized by low compliance and a second compound characterized by high compliance for use in enhancing the compliance of the first compound.

In some embodiments, the modified release delivery system comprises a composition comprising a first treatment (first compound) for use in treating, preventing, inhibiting, reducing, eliminating, protecting or delaying the onset of a chronic ophthalmic diseases, specifically, these associated with need to reduce IOP and a second compound capable of treating, preventing, inhibiting, reducing, eliminating, protecting or delaying the onset of an ocular condition having mid or severe symptoms.

In some embodiments, the modified release delivery system comprises a composition comprising a first treatment (first compound) for use in treating, preventing, inhibiting, reducing, eliminating, protecting or delaying the onset of glaucoma and a second compound capable of treating, preventing, inhibiting, reducing, eliminating, protecting or delaying the onset of an ocular condition having mid or severe symptoms.

The term modified-release as used herein refers to a system that alter the timing and/or the rate of release of a compound.

In some embodiments, the modified release is one or more of extended release, delayed release, controlled release sustained release, or repeated action.

In some embodiments, the delivery system allows a modified or controlled release mode selected from extended release, slow release, pulse release, or sustained release of the composition (the at least one first compound and the at least one second compound) at the eye.

Extended release as used herein refers to a dosage consisting of a sustained release or controlled release dosage.

Sustained release as used herein refers to a system provides an immediate release followed by a gradual release over time that maintains release of a compound over a specified period but not necessarily at a constant rate.

Controlled release as used herein refers to a system which release a compound over a specified period at a nearly constant rate.

In some embodiments the composition comprises ingredients that induce or affect a controlled release mode (controlled release ingredients) of any one or combination of the first or second compounds.

The controlled release ingredients may be selected from lipids, cholesterol, anionic, cationic and non ionic surfactants, cations, hyaluronic acid, celluloses, acrylates, vegetable derived oils, cationic and anionic polymers such as, polylactide, glycolide, and mineral salts such as mineral acids selected from phosphoric acid, hydrochloric acid and nitric acid

In some embodiments, the delivery system is one or more of an emulsion, an ointments, a suspension, an aqueous gel, a nanomicelle, a nanoparticle, a liposome, a dendrimer, an implant, contact lenses, a nanosuspension, a microneedle, in situ thermosensitive gel or any combination thereof.

In some embodiments, the composition is in a form of an emulsion. An emulsion typically offers an advantage to improve both solubility and bioavailability of the first compound and/or the second compound.

In some embodiments, the emulsion is an oil in water (o/w) emulsion or water in oil (w/o) emulsion. In some other embodiments, the emulsion is an o/w emulsion.

In some embodiment, the composition is an ophthalmic ointment. As appreciated, ointment typically have a greater retention within the precorneal region and improves ocular bioavailability and sustain the drug release.

Ocular ointment comprises of mixture of semisolid and a solid hydrocarbon (paraffin) that has a melting point at physiological ocular temperature (e.g. 34° C.). The choice of hydrocarbon is dependent on biocompatibility.

Ophthalmic ointments are generally prepared by dispersion of a compound in the pre-prepared ointment base. Non-limiting examples of an ointment base include, hydrocarbon bases, such as paraffin mixtures. The ointment may include additional excipients.

In some embodiments, the composition comprises a nanocarrier. In some embodiments, the nanocarrier is a nanoparticle, a nanosuspension, a liposome, a nanomicelle, a dendrimer or any combination thereof.

In some embodiments, the first compound, the second compound or both are embedded (entrapped) within a nanomicelle. Nanomicelles are typically made with amphiphilic molecules, which may be surfactant or polymeric in nature.

In some embodiments, the first compound, the second compound or both are embedded (entrapped) within a nanoparticle. Nanoparticles are colloidal carriers with a size range of 10 to 1000 nm.

In some embodiments, the nanoparticles are composed of lipids, proteins, natural or synthetic polymers.

In some embodiments, the nanoparticles are composed of albumin, sodium alginate, chitosan, poly (lactide-co-glycolide) (PLGA), polylactic acid (PLA), polycaprolactone or nay combination thereof.

In some embodiments, the nanoparticles are nanocapsules or nanospheres.

In some embodiments, the first compound, the second compound or both may be embedded or entrapped within a polymeric matrix.

The polymeric matrix in the context of the present disclosure may be biocompatible i.e. is inert to body tissue, such that upon administration to a body, it will not be toxic, injurious, physiologically reactive or cause any immunological rejection of the composition of matter. The polymeric matrix is also a water absorbing matrix and in the context of the present disclosure is absorbed or can absorb water, thereby forming a gel or a hydrogel.

The polymer(s) forming the matrix can be a naturally occurring polymer or a synthetic or semi-synthetic polymer. In some examples, the matrix forms a hydrogel that is a thermal responsive cross-linked hydrogel.

In some embodiments, the first compound and the second compound are enclosed within a nanocapsule. In some embodiments, the first compound and the second compound are enclosed (entrapped) inside a polymeric shell.

In some embodiments, the first compound and the second compound are within a nanosphere. In some embodiments, the first compound and the second compound are uniformly distributed throughout a polymeric matrix.

In some embodiments, the pharmaceutical composition comprises liposomes. The liposomes may be used for local delivery of the compounds described herein, preferably, for local controlled delivery. The liposomes comprise at least one liposome forming lipid, which forms the liposomes' membrane. The liposomes' membrane is a bilayer membrane and may be prepared to include a variety of physiologically acceptable liposome forming lipids and, as further detailed below, non-liposome forming lipids (at the mole ratio which support the formation and maintenance of stable liposomes).

As used herein, the term “liposome forming lipids” is used to denote primarily glycerophospholipids and sphingomyelins which when dispersed in aqueous media by itself at a temperature above their solid ordered to liquid disordered phase transition temperature will form stable liposomes. Examples of liposome forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS).

In some embodiments, the composition is in a form of a dendrimer. As used herein a dendrimer is characterized as nanosized, highly branched, star shaped polymeric systems. In some embodiments, the dendrimer is a poly (amidoamine) (PAMAM) dendrimer.

The delivery system described herein can be configured or adjusted or tuned to control the release of the first compound and the second compound.

In some embodiments, the delivery system is configured to allow simultaneous release of the first compound and second compounds.

In some other embodiments, the delivery system is configured to allow modified release of at least one of the first compound or the second compound sequentially.

In some other embodiments, the delivery system is configured to allow each one of the first compound and second compound to have an individual release rate.

In some further embodiments, the delivery system is configured to allow each of the first compound and the second compound independently from the other to have extended release, slow release, pulse release, or sustained release profile.

The delivery system can be prepared by any method known in the art and may include any vehicle, for example, a polymeric matrix.

In some embodiments the composition is comprised within a delivery system selected from particulate, nanoparticles, liposomes, emulsions, and contact lens.

In accordance with some aspects which can be implemented as embodiments of the delivery system of the invention, the delivery system allowing modified release comprises liposomes comprising at least one PGA or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising one or more of latanoprost (isopropyl analogue of PGF2α), bimatoprost, travoprost, tafluprost, latanoprostene bunod, any pharmaceutically acceptable salt thereof, solvate thereof, hydrate thereof, any stereoisomer therefore, physiologically functional derivative thereof or combinations thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising one or more of latanoprost, travoprost, tafluprost, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising latanoprost, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising tafluprost, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising travoprost, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising bimatoprost, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising latanoprostene bunod, any pharmaceutically acceptable salt thereof, solvate thereof, hydrate thereof, any stereoisomer therefore, physiologically functional derivative thereof or combination thereof and pilocarpine or any pharmaceutically acceptable salt thereof, solvate thereof, hydrate thereof, any stereoisomer therefore, physiologically functional derivative thereof.

In accordance with some aspects which can be implemented as embodiments of the delivery system of the invention, the delivery system allowing modified release comprises liposomes comprising nitric oxide and pilocarpine or any pharmaceutically acceptable salt thereof, solvate thereof, hydrate thereof, any stereoisomer therefore, physiologically functional derivative thereof.

Exemplary compositions are provided in Example 1 below.

Accordingly, in some embodiments, the composition comprises latanoprost or any pharmaceutically acceptable salt thereof, solvate thereof, hydrate thereof, any stereoisomer therefore, physiologically functional derivative thereof, pilocarpine or any pharmaceutically acceptable salt thereof, solvate thereof, hydrate thereof, any stereoisomer therefore, physiologically functional derivative thereof, cyclodextrin, a surfactant, a buffer, a thickener and a preservative.

The amounts of each one of latanoprost and pilocarpine can be adjusted to allow effective treatment.

In some embodiments, the composition comprises latanoprost or any pharmaceutically acceptable salt thereof, solvate thereof, hydrate thereof, any stereoisomer therefore, physiologically functional derivative thereof, pilocarpine or any pharmaceutically acceptable salt thereof, solvate thereof, hydrate thereof, any stereoisomer therefore, physiologically functional derivative thereof, chitosan, a surfactant, a buffer and a thickener.

In some embodiments, the composition comprises latanoprost or any pharmaceutically acceptable salt thereof, solvate thereof, hydrate thereof, any stereoisomer therefore, physiologically functional derivative thereof, pilocarpine or any pharmaceutically acceptable salt thereof, solvate thereof, hydrate thereof, any stereoisomer therefore, physiologically functional derivative thereof, Particle/PLGA, a surfactant and a buffer.

In some embodiments, the composition comprises latanoprost or any pharmaceutically acceptable salt thereof, solvate thereof, hydrate thereof, any stereoisomer therefore, physiologically functional derivative thereof, pilocarpine or any pharmaceutically acceptable salt thereof, solvate thereof, hydrate thereof, any stereoisomer therefore, physiologically functional derivative thereof, at least one hydrophobic polymer (e.g. ethylcellulose hydrophilic hydroxypropyl methyl cellulose), a surfactant and a buffer.

In some embodiments, the composition is at least one composition denoted in Example 1 as one or more of formulation 1 to formulation 26.

The composition may be administered by appropriate administration/schedule regimen. In some embodiments, the composition is to be administrated at least twice daily, at least once daily, at least once in two days, or at least once a week.

As detailed herein, the present disclosure also provides in accordance with some aspects, a kit comprising at least one first compound capable of treating a condition associated with low compliance and a second compound capable of treating a different condition having mild or severe symptoms.

In some embodiments, the kit comprises at least one first compound capable of treating a condition associated with low compliance and a second compound capable of treating a different condition associated with high compliance.

In some embodiments, the kit comprises a first compound capable of treating, preventing, inhibiting, reducing, eliminating, protecting or delaying the onset of a chronic ophthalmic diseases, specifically, these associated with need to reduce IOP and a second compound capable of treating, preventing, inhibiting, reducing, eliminating, protecting or delaying the onset of an ocular condition having mid or severe symptoms.

In some embodiments, the kit comprises a first compound capable of treating, preventing, inhibiting, reducing, eliminating, protecting or delaying the onset of glaucoma and a second compound capable of treating, preventing, inhibiting, reducing, eliminating, protecting or delaying the onset of an ocular condition having mid or severe symptoms.

In some embodiments, the kit comprises a first component and a second component, the first component comprises the at least one first compound and the second component comprises the at least one second compound.

In some embodiments, the first component and/or the second component is provided in a unit dose for example a distinct pharmaceutical composition. Namely the kit comprises each compound (i.e. the first compound and the second compound) in a discrete unit. The unit dose can contain an effective amount—a prescribed quantity of the first compound and the second compound sufficient to produce a therapeutic effect.

In some embodiments, the two compounds are packed each in discrete unit dose; namely in a discrete unit which is packaged and is suitable for administration to the subject (human or non-human) Each unit dose may be for example an individual ampoule or a syringe.

For example, a kit may comprise formulation #1 and formulation #2 of example 1, each in discrete units and the two formulations mixed prior to administration to form formulation #3.

In some embodiments, the kit comprises instructions for combining the two pharmaceutical dosage forms (unit dose) one into the other to form a composition.

In some embodiments, the kit comprises a container and instructions to combine the two pharmaceutical dosage forms into the container.

In some embodiments, the kit comprises as single component (i.e. unit dose) comprising the at least one first compound and the at least one second compound.

In some embodiments, the kit comprises a single composition comprising the two compounds.

In some embodiments, the kit includes a dropper nozzle or a tube with a nozzle. In some embodiments, the kit comprises a dropper.

In some embodiments, the kit comprises instructions for use in treating glaucoma.

In some embodiments, the kit comprises instructions for administration by appropriate administration/schedule regimen. In some embodiments, the instructions include administration the composition at least twice daily, at least once daily, at least once in two days, or at least once a week.

As described herein, the present disclosure also provides a method for enhancing therapeutic compliance of a low compliance treatment, such as a treatment directed to an ophthalmic disease. Hence, it is provided a method for enhancing compliance of treatment of an ophthalmic disease, the method comprising administering to an individual in need thereof, a pharmaceutical composition comprising an effective amount of at least one first compound capable of reducing IOP, and an effective amount of at least one second compound capable of treating, preventing, inhibiting, reducing, eliminating, protecting or delaying the onset of an ocular condition having mild or severe symptoms.

In some embodiments, the first compound is capable of treating, preventing, inhibiting, reducing, eliminating, protecting or delaying the onset of a condition that requires reduction IOP. Hence, in accordance with some aspects it is provided, a method for enhancing compliance of treatment of an ophthalmic disease, the method comprising administering to an individual in need thereof, a pharmaceutical composition comprising an effective amount of at least one first compound capable of treating, preventing, inhibiting, reducing, eliminating, protecting or delaying the onset of a condition that requires reduction IOP, and an effective amount of at least one second compound capable of treating, preventing, inhibiting, reducing, eliminating, protecting or delaying the onset of an ocular condition having mild or severe symptoms.

In accordance with some aspects, which may be implemented as embodiments of the methods, it is provided a method of treating, preventing, inhibiting, reducing, eliminating, protecting or delaying the onset of a condition that requires reduction IOP, the method comprising administering to an individual in need thereof, a pharmaceutical composition comprising an effective amount of at least one first compound capable of treating, preventing, inhibiting, reducing, eliminating, protecting or delaying the onset of a condition that requires reduction IOP, and an effective amount of at least one second compound capable of treating, preventing, inhibiting, reducing, eliminating, protecting or delaying the onset of an ocular condition having mild or severe symptoms.

In some embodiments, the condition that requires reduction IOP is glaucoma.

In some specific embodiments, the methods of the invention provide a method for enhancing compliance of an anti-glaucoma treatment, the method comprising administering to an individual in need thereof, a pharmaceutical composition comprising an effective amount of at least one first compound capable of treating, preventing, inhibiting, reducing, eliminating, protecting or delaying the onset of glaucoma, and an effective amount of at least one second compound capable of treating, preventing, inhibiting, reducing, eliminating, protecting or delaying the onset of an ocular condition having mild or severe symptoms.

In some specific embodiments, the methods of the invention provide a method of treating, preventing, inhibiting, reducing, eliminating, protecting or delaying the onset of glaucoma, the method comprising administering to an individual in need thereof, a pharmaceutical composition comprising an effective amount of at least one first compound capable of treating, preventing, inhibiting, reducing, eliminating, protecting or delaying the onset of glaucoma, and an effective amount of at least one second compound capable of treating, preventing, inhibiting, reducing, eliminating, protecting or delaying the onset of an ocular condition having mild or severe symptoms.

In some embodiments, the methods of the invention comprise administering to an individual in need thereof, a pharmaceutical composition comprising at least one PGA or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising one or more of latanoprost, bimatoprost, travoprost, tafluprost, latanoprostene bunod, any pharmaceutically acceptable salt thereof, solvate thereof, hydrate thereof, any stereoisomer therefore, physiologically functional derivative thereof or combinations thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising latanoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising tafluprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising travoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising bimatoprost or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

In some embodiments, the methods of the invention comprise administering to an individual in need thereof, a pharmaceutical composition comprising latanoprostene bunod or any pharmaceutically acceptable salt thereof, solvate thereof, hydrate thereof, any stereoisomer therefore, physiologically functional derivative thereof and pilocarpine or any pharmaceutically acceptable salt thereof, solvate thereof, hydrate thereof, any stereoisomer therefore, physiologically functional derivative thereof.

n some embodiments, the methods of the invention comprise administering to an individual in need thereof, a pharmaceutical composition comprising at least nitric oxide or any pharmaceutically acceptable salt thereof, solvate thereof, hydrate thereof, any stereoisomer therefore, physiologically functional derivative thereof.

In some embodiments, the method comprising administrating the composition into the eye. In some embodiments, the method comprising the composition at least twice daily, at least once daily, at least once in two days, or at least once a week.

As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith.

The term “treatment, treat, treating” as used herein mean ameliorating one or more clinical indicia of disease activity by administering a pharmaceutical composition or the kit of the invention in a patient having a chronic ophthalmic disease, specifically those associated with the need to reduce IOP, such that are characterized by increased intraocular pressure, such as glaucoma.

As described herein, the enhanced compliance of the first compound may be measured by treatment success, specifically, by ameliorating undesired symptoms associated with a chronic ophthalmic disease, to prevent the manifestation of such symptoms before they occur, to slow down the progression of the chronic ophthalmic disease, slow down the deterioration of symptoms, slow down the irreversible damage caused in the progressive chronic stage of the disease, to delay the onset of said progressive stage, to lessen the severity or cure the chronic ophthalmic disease, to enable more rapid recovery, or to prevent the chronic ophthalmic disease form occurring or a combination of two or more of the above.

The terms “inhibition”, “reduction”, “decrease” or the like as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

The terms “increase”, “enhance”, or the like as referred to herein, relate to the increase or enhancement of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

Specifically, in the context of the present invention, inhibition or reduction in the condition to be treated by the first treatment as a consequence of the enhanced compliance of the composition and kits of the invention relate to reduction of IOP of between about 5% and about 90%, between about 10% and about 80%, between about 10% and about 70%, between about 10% and about 60%, between about 10% and about 50%, between about 10% and about 40%.

The present invention relates to the treatment of subjects, or patients, in need thereof. By “patient” or “subject in need” it is meant any organism to whom the composition/s, kit/s, and methods herein described is desired, including humans and domestic mammals. In some specific embodiments, the treated subject may be a human subject.

The term “effective amount” means an amount necessary to achieve a selected result and may be determined by the severity of the disease in conjunction with the preventive or therapeutic objectives, the route of administration and the patient's general condition (age, sex, weight and other considerations known to the attending physician).

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### NON-LIMITING EXAMPLES

### Example 1

### Preparation of Compositions

The below listed formulations were prepared by the following steps:
(i) Solubilizing active ingredient(s) (compound) with cyclodextrin and surfactant;
(ii) Adding the remaining excipients; and optionally
(iii) Adding preservative (if product subsequently sterilized).

TABLE 1
Cyclodextrin and Surfactant with Polymer (formulations #1-#3)

| | Formulation 1 | Formulation 2 | Formulation 3 |
|---|---|---|---|
| Ingredient | % range | | |
| Pilocarpine | 0.1 to 6 | | 0.1 to 6 |
| Latanoprost | | 0.005 to 2 | 0.005 to 2 |
| Cyclodextrin (various) | 0.1 to 5 | 0.1 to 5 | 0.1 to 5 |
| Surfactant (e.g. | 0.1 to 5 | 0.1 to 5 | 0.1 to 5 |
| poloxamer) | | | |
| Water | <90 | <90 | <90 |
| Buffer (pH 5-7.8)/ | 0.1 to 4 | 0.1 to 4 | 0.1 to 4 |
| EDTA) | | | |
| Thickener | 0.1 to 2 | 0.1 to 2 | 0.1 to 2 |
| Preservative (e.g. | 0.2 | 0.2 | 0.2 |
| Benzalkonium | | | |
| chloride) | | | |

TABLE 2
Chitosan & Surfactant with Polymer (formulations #4-#6)

| | Formulation 4 | Formulation 5 | Formulation 6 |
|---|---|---|---|
| Ingredient | % range | | |
| Pilocarpine | 0.1 to 6 | | 0.1 to 6 |
| Latanoprost | | 0.005 to 2 | 0.005 to 2 |
| Chitosan | 0.1 to 5 | 0.1 to 5 | 0.1 to 5 |
| Surfactant | 0.1 to 5 | 0.1 to 5 | 0.1 to 5 |
| Water | <90 | <90 | <90 |
| Buffer pH 5-7.8/ | 0.1 to 4 | 0.1 to 4 | 0.1 to 4 |
| EDTA | | | |
| Thickener | 0.1 to 2 | 0.1 to 2 | 0.1 to 2 |

TABLE 3
Hyaluronic Polymer (formulations #7-#9)

| | Formulation 7 | Formulation 8 | Formulation 9 |
|---|---|---|---|
| Ingredient | % range | | |
| Pilocarpine | 0.1 to 6 | | 0.1 to 6 |
| Latanoprost | | 0.005 to 2 | 0.005 to 2 |
| Hyaluronic acid | 0.1 to 5 | 0.1 to 5 | 0.1 to 5 |
| Surfactant | 0.1 to 5 | 0.1 to 5 | 0.1 to 5 |
| Water | <90 | <90 | <90 |
| Buffer (pH 5-7.8)/ | 0.1 to 4 | 0.1 to 4 | 0.1 to 4 |
| EDTA) | | | |
| Thickener | 0.1 to 2 | 0.1 to 2 | 0.1 to 2 |

TABLE 4
Polymer/Particle PLGA (formulations #10-#12)

| | Formulation 10 | Formulation 11 | Formulation 12 |
|---|---|---|---|
| Ingredient | % range | | |
| Pilocarpine | 0.1 to 6 | | 0.1 to 6 |
| Latanoprost | | 0.005 to 2 | 0.005 to 2 |
| Particle/PLGA | 0.2 to 5 | 0.1 to 5 | 0.1 to 5 |
| Surfactant | 0.1 to 5 | 0.1 to 5 | 0.1 to 5 |
| Water | <90 | <90 | <90 |
| Buffer (pH 5-7.8)/ | 0.2 to 4 | 0.1 to 4 | 0.1 to 4 |
| EDTA) | | | |

TABLE 5
Polymer/Polymeric film (formulations #13-#15)

| | Formulation 13 | Formulation 14 | Formulation 15 |
|---|---|---|---|
| Ingredient | % range | | |
| Pilocarpine | 0.1 to 6 | | 0.1 to 6 |
| Latanoprost | | 0.005 to 2 | 0.005 to 2 |
| Ethyl cellulose or | 0.3 to 5 | 0.1 to 5 | 0.1 to 5 |
| Carbopol | | | |
| hydroxypropyl | 0.1 to 5 | 0.1 to 5 | 0.1 to 5 |
| cellulose | | | |
| Surfactant | 0.1 to 5 | 0.1 to 5 | 0.1 to 5 |
| Water | 0.4 <90 | <90 | <90 |
| Buffer (pH 5-7.8)/ | 0.1 to 2 | 0.1 to 2 | 0.1 to 2 |
| EDTA) | | | |

TABLE 6
Liposome (formulations #16-#18)

| | Formulation 16 | Formulation 17 | Formulation 18 |
|---|---|---|---|
| Ingredient | % range | | |
| Pilocarpine | 0.1 to 6 | | 0.1 to 6 |
| Latanoprost | | 0.005 to 2 | 0.005 to 2 |
| Dipalmitoylphos- | 0.1 to 5 | 0.1 to 5 | 0.1 to 5 |
| phatidylcholine | | | |
| Phosphati- | 0.1 to 5 | 0.1 to 5 | 0.1 to 5 |
| dylethanolamine | | | |
| Cholesterol | 0.1 to 2 | 0.1 to 2 | 0.1 to 2 |
| Surfactant | 0.1 to 5 | 0.1 to 5 | 0.1 to 5 |
| Water | <90 | <90 | <90 |
| Buffer (pH 5-7.8)/ | 0.1 to 2 | 0.1 to 2 | 0.1 to 2 |
| EDTA) | | | |

TABLE 7
Emulsion (formulations #19-#21)

| | Formulation 19 | Formulation 20 | Formulation 21 |
|---|---|---|---|
| Ingredient | % range | | |
| Pilocarpine | 0.1 to 6 | | 0.1 to 6 |
| Latanoprost | | 0.005 to 2 | 0.005 to 2 |
| Oil (mineral or | 1 to 30 | 1 to 30 | 1 to 30 |
| natural) | | | |
| Propylene Glycol | 2 to 20 | 2 to 20 | 2 to 20 |
| Surfactants | 0.1 to 5 | 0.1 to 5 | 0.1 to 5 |
| (e.g. poloxamer) | | | |
| Water | <90 | <90 | <90 |
| Buffer (pH 5-7.8)/ | 0.2 to 2 | 0.1 to 2 | 0.1 to 2 |
| EDTA) | | | |
| Thickener | 0.3 to 2 | 0.1 to 2 | 0.1 to 2 |
| Preservative (e.g. | 0.3 | 0.2 | 0.2 |
| Benzalkonium | | | |
| chloride) | | | |

TABLE 8
Dendrimer (formulations #22-#24)

| | Formulation 22 | Formulation 23 | Formulation 24 |
|---|---|---|---|
| Ingredient | % range | | |
| Pilocarpine | 0.1 to 6 | | 0.1 to 6 |
| Latanoprost | | 0.005 to 2 | 0.005 to 2 |
| Dendrimer | 0.1 to 10 | 0.1 to 10 | 0.1 to 10 |
| Propylene Glycol | 0.5 to 20 | 0.5 to 20 | 0.5 to 20 |
| Surfactant(s) | 0.1 to 5 | 0.1 to 5 | 0.1 to 5 |
| Water | <90 | <90 | <90 |
| Buffer (pH 5-7.8)/ | 0.1 to 2 | 0.1 to 2 | 0.1 to 2 |
| EDTA) | | | |
| Preservative (e.g. | 0.4 | 0.2 | 0.2 |
| Benzalkonium | | | |
| chloride) | | | |

TABLE 9
Separate manufacture and combine at point of use in single syringe

| | |
|---|---|
| Formulation 25 | Formulation 26 |

| | |
|---|---|
| Ingredient | % range |

| | | |
|---|---|---|
| Pilocarpine | 0.1 to 6 | |
| Latanoprost | | 0.005 to 2 |
| Cyclodextrin | 0.5 to 5 | |
| Chitosan | | 0.1 to 5 |
| Surfactant | 0.1 to 5 | 0.1 to 5 |
| Water | <90 | <90 |
| Buffer (pH 5-7.8)/ | 0.1 to 2 | 0.1 to 2 |
| EDTA | | |

1-33. (canceled)

34. A pharmaceutical composition comprising:
(i) a prostaglandin analogue (PGA), or a pharmaceutically acceptable salt thereof, solvate thereof, hydrate thereof, any stereoisomer therefore, physiologically functional derivative thereof; and
(ii) between about 0.1% w/w to about 3% w/w pilocarpine, or a pharmaceutically acceptable salt thereof, solvate thereof, hydrate thereof, stereoisomer thereof, or physiologically functional derivative thereof.

35. The pharmaceutical composition of claim 34, comprising between about 0.1% w/w to about 1% w/w pilocarpine, or a pharmaceutically acceptable salt thereof, solvate thereof, hydrate thereof, stereoisomer thereof, or physiologically functional derivative thereof.

36. The pharmaceutical composition of claim 34, wherein the PGA is latanoprost, or a pharmaceutically acceptable salt thereof, solvate thereof, hydrate thereof, any stereoisomer therefore, physiologically functional derivative thereof.

37. The pharmaceutical composition of claim 36, comprising from 0.005% w/w to 2% w/w latanoprost.

38. The pharmaceutical composition of claim 34, further comprising a carrier, excipient or additive.

39. The pharmaceutical composition of claim 34, wherein the pharmaceutical composition is an ophthalmic ointment.

40. A method of reducing intra-ocular pressure in an individual in need thereof, the method comprising administering to the individual the pharmaceutical composition of claim 34.

41. A method of treating glaucoma in an individual in need thereof, the method comprising administering to the individual the pharmaceutical composition of claim 34.

42. A method of treating presbyopia in an individual in need thereof, the method comprising administering to the individual the pharmaceutical composition of claim 34.
